(19) United States
(12) Patent Application Publication
(10) Pub. No.: US 2006/0286631 A1
(43) Pub. Date: Dec. 21, 2006

(54) Method for preparating t-20 peptide by high cell density cultivation of recombinant e. coli containing t-20 peptide coding gene

(76) Inventors: Choong Kyung Kang, Gyeonggi-do (KR); Nam Hee Choi, Seoul (KR); Jae Wok Yoo, Gyeonggi-do (KR); Keon Tae Tak, Incheon (KR); Hong Ki Jang, Incheon (KR)

Correspondence Address: J.C. Patents, 4 Venture, Suite 250, Irvine, CA, 92618, US

(21) Appl. No.: 10/570,217

(22) PCT Filed: Aug. 30, 2004
(86) PCT No.: PCT/KR04/02182

§ 371 Date: Feb. 28, 2006

(30) Foreign Application Priority Data
Sep. 1, 2003 (KR) 10-2003-0060820

(51) Int. Cl. C12P 21/06; C07K 14/16; C07H 21/04; C12N 15/74; C12N 1/21

(52) U.S. Cl. 435/69.1; 435/252.33; 435/488; 530/350; 536/23.7

(57) ABSTRACT

The present invention relates to a method for preparing a T-20 peptide by the high-cell-density cultivation of recombinant E. coli containing a T-20 peptide-coding gene, as well as a method for isolating and purifying the T-20 peptide with high purity from E. coli where the T-20 peptide have been expressed. According to the present invention, the recombinant E. coli containing the T-20 peptide-coding gene can be cultured at a high cell concentration, and the T-20 peptide can be isolated and purified with high purity from said recombinant E. coli, thereby making it possible to obtain the inventive T-20 peptide with high industrial value economically.

### TECHNICAL FIELD

The present invention relates to a method for preparing a T-20 peptide by the high-cell-density cultivation of recombinant E. coli containing a T-20 peptide-coding gene. More particularly, the present invention relates to a method for expressing a T-20 peptide by the high-cell-density cultivation of said recombinant E. coli containing a T-20 peptide-coding gene using a fed-batch culture process (pH-stat) in which nutrient medium is cumulatively added so as to maintain the pH of a culture broth of said recombinant E. coli at a suitable level, as well as a method for isolating and purifying the T-20 peptide with high purity by solvent treatment of recombinant E. coli where the T-20 peptide have been expressed.

### BACKGROUND ART

T-20 peptide is a peptide with the amino acid sequence of residues 638-673 of gp41 which is a HIV-1_{LA1} transmembrane protein, and is known to act to inhibit HIV infection into CD-4⁺ cells. The structure and function of the T-20 peptide are already well known by U.S. Pat. Nos. 5,464,933 and 5,656,480. Particularly, a chemical synthetic method for preparing the T-20 peptide is already disclosed in U.S. Pat. No. 6,015,881.

A typical chemical synthetic method of peptides is a process which is very complicated and requires high costs. Particularly, in order to chemically synthesize certain peptides, a process, of binding and purifying the desired amino acids in a solid or liquid phase according to the desired order, must be repeatedly performed. Namely, highly purified amino acids or their derivatives must be used as a raw material, and as the number of amino acid residues forming the desired peptide increases, a production process becomes complex, the yield decreases and production cost increases exponentially. For this reason, even if industrially useful peptides were developed, their industrial application encounters difficulties due to a complication in production processes and a reduction in economic efficiency.

Korean Patent 263583, U.S. Pat. No. 6,183,992 and Korean Patent 319529 disclose a method for biologically producing useful peptides using microorganisms. The biological production method of useful peptides utilizes naturally occurring, relatively inexpensive, eco-friendly medium components as main materials, without using, as raw materials, highly purified amino acids or certain peptide fragments required in a chemical production method. Thus, the biological production method has advantages in that it allows low production costs due to inexpensive raw materials and does not cause a problem in the supply of raw materials. Other advantages are that it allows a relatively short time period for microbial cultivation as a main process, (e.g., less than 72 hours for E. coli), and can produce peptides with high purity without great difficulty by a process of isolating and purifying the desired fusion peptides.

Among the biological production methods of useful peptides, methods known to use microorganisms include batch culture, continuous culture and fed-batch culture methods. In the comparison among these methods, the batch culture method has a shortcoming that it has a lower productivity than that of the continuous method, whereas the continuous culture method is disadvantageous in that it has a possibility for bacterial cells to be contaminated due to a long culture time period and requires high equipment investment costs, thus making it difficult to industrially apply the continuous batch method. On the other hand, the fed-batch culture method has advantages in that can increase productivity as compared to the batch culture method and is easy to apply industrially as compared to the continuous culture mode. Due to such advantages, many studies on the fed-batch culture method and many attempts to industrially apply the fed-batch culture method are being recently conducted.

Meanwhile, in a fermentation process for obtaining microbial cells themselves as a final product, an increase in the content of bacterial cells provides advantages, but there are some limitations in increasing the content of bacterial cells to the maximum. In other words, if a fermenter is filled only with microbial cells having some water content (70-80%), the maximum limit on the content of microbial cells will theoretically be about 240˜280 g/L but it will vary depending on the kind of microorganisms. However, in practice, nutrients must be suitably fed for microbial cultivation and if the content of microbial cells reaches a certain level, mass transfer becomes impossible and cell growth stops, and an ultimate cell density at which microorganisms can be cultured becomes lower than that level. In a process of preparing useful substances by microbial cultivation, the productivity of useful substances can be increased only by either increasing the cell density or increasing the content of useful substances. Thus, the high cell density culture method is highly useful for the improvement in productivity of useful substances.

### DISCLOSURE OF INVENTION

Accordingly, the present inventors have conducted studies to perform the high-cell-density cultivation of recombinant E. coli containing a gene encoding a commercially useful T-20 peptide, and at the same time, to obtain the T-20 peptide with high purity from the cultured recombinant E. coli where the T-20 peptide have been expressed. As a result, the present inventors have developed a method of culturing cells having the T-20 peptides expressed, at a high cell density, using a fed-batch culture process in which the largest possible amount of microbial cells can be obtained by optimizing culture conditions in order that a given cell density of recombinant E. coli can continue to grow, as well as a method of efficiently isolating and purifying the T-20 peptide using acetone. On the basis of these developments, the present invention has been perfected.

Therefore, it is an object of the present invention to provide a method for preparing the T-20 peptide by the high-cell-density culture of recombinant E. coli containing a T-20 peptide-coding gene.

Another object of the present invention is to provide a method for isolating and purifying the T-20 peptide with high purity from the cultured recombinant E. coli.

To achieve the above objects, in one aspect, the present invention provides a method for preparing a T-20 peptide by the high-cell-density culture of recombinant E. coli containing a T-20 peptide-coding gene, the method comprising the steps of: (a) batch-culturing recombinant E. coli containing the T-20 peptide coding gene in an initial culture medium (pH 6.8-7.2) containing mineral solution; (b) culturing the recombinant E. coli culture while adding a nutrient medium automatically so as to adjust the pH of the culture broth to 6.8-7.2; and (c) adding an expression inducer when the absorbance (OD₆₀₀) of the recombinant E. coli culture broth reaches 30-100, and culturing the recombinant E. coli additionally so as to induce the expression of the T-20 peptide.

In another aspect, the present invention provides a method for isolating and purifying a T-20 peptide, the method comprising the steps of: (a) collecting the E. coli where the T-20 peptide have been expressed in a form fused with a fusion partner by the method of the first aspect, and then suspending the collected E. coli in distilled water; (b) treating the recombinant E. coli suspension with an organic solvent, to obtain a precipitate containing a fusion protein in the form of the T-20 peptide-fusion partner; (c) cutting out the fusion partner from the fusion protein, and then treating the remaining protein with an organic solvent so as to obtain a precipitate containing the T-20 peptide; and (d) subjecting the precipitate to chromatography so as to remove impurities, treating the remaining protein with an organic solvent, and collecting the treated protein. In the present invention, the step (c) preferably comprises the sub-steps of: (i) adsorbing the precipitate solution onto a Sepharose column, eluting the Sepharose column at least two times with a concentration gradient of sodium chloride, collecting the resulting precipitate, and precipitating the precipitate by treating with acetone; and (ii) subjecting the precipitate obtained in the step (i) to C18 column chromatography so as to remove impurities, and precipitating the remaining substance by treatment with acetone.

Hereinafter, the present invention will be described in detail.

In the present invention, recombinant E. coli producing the T-20 peptide was cultured at a high cell density by a fed-batch culture method wherein (1) a nutrient medium was additionally fed during the cultivation of E. coli using a pH-stat method with excellent process convenience; (2) temperature, pH, stirring rate, etc., were maintained constant, such that the physiological activity of cells would not be inhibited even at a higher cell concentration than a given cell concentration per fermentation volume; (3) culture conditions were suitably controlled such that dissolved oxygen would not be limited. Then, the T-20 peptide expressed in the recombinant E. coli was isolated and purified with high purity using acetone.

The recombinant E. coli which is used for the mass production of the T-20 peptide in the present invention preferably contains not only the T-20 peptide-coding gene but also a lactose-inducible promoter. Furthermore, the recombinant E. coli, as a fusion partner which is expressed in a form fused with T-20 peptide, preferably contains a gene encoding the fusion partner which prevents peptide or protein from being degraded by intracellular protease.

Meanwhile, the T-20 peptide-coding gene may be easily selected and used from already known genes deposited at gene sequence databases, such as NCBI GenBank. The recombinant E. coli containing the T-20 peptide-coding gene may be suitably selected by any person skilled in the art, and it may preferably be E. coli BmG3 strain (KCCM 10506), but not limited thereto.

In the present invention, in order to induce the expression of the target T-20 peptide in E. coli cells, lactose, which is an expression inducer is further added when the absorbance (OD₆₀₀) of the recombinant E. coli culture broth is about 30-100. The additional amount of lactose is preferably 6-8% (w/v) per total culture broth, but not limited thereto.

In the present invention, the T-20 peptide produced by the recombinant E. coli containing the T-20 peptide-coding gene includes both a final product itself expressed from the E. coli, and an intermediate of the T-20 peptide.

To effectively extract, isolate and purify the T-20 peptide expressed in the recombinant E. coli which had been sufficiently cultured by the inventive fed-batch culture method, C18 high-pressure liquid chromatography or Sepharose resin is preferably used, but not limited thereto. The organic solvent used is preferably a ketone solvent, and more preferably acetone or acetonitrile. The acetone solvent which is an inexpensive industrial solvent is economically advantageous and also has the advantage of an excellent extraction effect. Particularly, according to the method of Example 2 in the present invention, the T-20 peptide can be purified with high purity an individual impurity content of less than 0.5% and a total impurity content of less than 2.0% by the acetone solvent.

A method of extracting an insoluble protein using acetone is well known, but there is no case of the use of acetone in the extraction of the T-20 fusion peptide, and the use of acetone as in below Example 2 is very effective since it allows the elimination of an intermediate step of purification. Namely, the treatment of isolated E. coli cells with the acetone solvent allows the disruption of cells together with easy extraction of the desired T-20 peptide in a fusion peptide form.

The T-20 peptide-bound fusion protein which had been extracted, isolated and purified by acetone according to the inventive method is treated with CNBr so as to cut off the fusion partner portion. Then, the T-20 peptide can be isolated with high purity using a commercially available column resin which had been suitably selected.

In the present invention, in order to maintain a cell growth state at optimal condition by controlling the pH of the recombinant E. coli culture containing the T-20 peptide-coding gene to be maintained at a suitable level during its cultivation, a nutrient medium was cumulatively added according to the changes in pH, so that the cell density of the E. coli, i.e., the dry weight of the recombinant E. coli obtainable per the volume of the culture broth, could be increased to 20-60 g per liter of the culture broth, and the absorbance (OD₆₀₀) of the culture broth could be increased to 100, thus increasing the production of the desired T-20 peptide. Furthermore, the T-20 peptide expressed in the E. coli cells was extracted and purified using inexpensive acetone so that the T-20 peptide with high purity could be efficiently prepared at large amounts in a short time period.

### BRIEF DESCRIPTION OF DRAWINGS

FIG. 1 is a graphic diagram showing the growth curve by fed-batch culture of recombinant E. coli containing a T-20 peptide-coding gene, wherein A represents cell dry weight, and B is absorbance (OD₆₀₀).

FIG. 2 is an electrophoresis photograph showing the expression of a T-20 peptide with the passage of time after the addition of lactose during fed-batch cultivation, wherein lanes 1-5 show results after 2, 4, 6, 8 and 10 hours, respectively.

FIG. 3 is an electrophoresis photograph according to each purification step, wherein lane 1: cultured bacterial cells; lane 2: after treatment with CNBr; lane 3: before column chromatography purification; and lane 4: after column chromatography purification.

FIG. 4 is a graphic diagram showing the results of HPLC analysis of purified T-20 peptide.

### DETAILED DESCRIPTION OF THE INVENTION AND PREFERRED EMBODIMENT THEREOF

The present invention will hereinafter be described in further detail by examples. It will however be obvious to a person skilled in the art that these examples can be modified into various different forms and the present invention is not limited to or by the examples. These examples are presented to further illustrate the present invention.

### Example 1

### High-Cell-Density Cultivation of Recombinant E. coli Containing T-20 Peptide

Recombinant E. coli BmG3 (KCCM 10506) containing the T-20 peptide was cultured under the following fermentation process conditions for fed-batch cultivation so as to produce a T-20 peptide in a form fused with a fusion partner. A fermenter having a total volume of 5 liters and allowing automatic adjustments to pH, temperature and bubble level was used to culture an E. coli culture at a total liquid volume of 3 liters. For this purpose, based on 1 liter of culture medium, 15 g of glucose, 4 g of ammonium sulfate, 3 g of potassium phosphate monobasic, 3 g of potassium phosphate dibasic, 1 g of sodium chloride, 2.3 g of sodium citrate, 5 mg of thiamine, 0.45 mg of magnesium sulfate, 50 μg of kanamycin and 0.2 g of antifoaming agent were added to distilled water. After the culture medium is sterilized, the recombinant E. coli BmG3 (KCCM 10506) was added at the amount of 2% of the culture medium so as to prepare 2.2 liters of E. coli-containing culture broth. Meanwhile, based on 1 liter of mineral solution, 11.5 g of calcium chloride, 7.3 g of iron citrate, 3.2 g of manganese chloride, 1.68 g of zinc acetate, 0.302 g of copper chloride, 0.534 g of cobalt chloride, 0.666 g of boric acid, 0.534 g of sodium molybdate, 0.534 g of thiamine, and 20 g of sodium citrate were added and 2N hydrochloric acid is added so as to prepare a mineral solution. 10 ml of the mineral solution was added to the culture medium, and the initial pH of the culture medium was adjusted to 6.8. Then, the culture medium was subjected to batch cultivation for about 7 hours until absorbance (OD₆₀₀) reaches 10.

Then, 200 ml of a nutrient medium prepared by dissolving 4 g of potassium phosphate monobasic, 2 g of potassium phosphate dibasic, 5 mg of thiamine, 4 g of ammonium sulfate and 50 μg of kanamycin in distilled water was added to the nutrient medium. Next, 500 ml of a solution prepared by dissolving 250 g of glucose and 4 g of magnesium sulfate in distilled water was added to the culture medium maintaining the pH of the culture broth at 6.8-7.2. At this time, 10 ml of the mineral solution prepared as described above was further added to the culture medium.

In the cultivation step as described above, the content within the fermenter was stirred with a stirrer equipped with two impellers, at 300-1,000 rpm, and the aeration rate of the culture medium was adjusted in order that the volume of air becomes 0.5-3 volumes per the volume of liquid within the fermenter per minute. The pH of the culture medium was maintained at about 6.8 using a pH-stat method by automatically feeding ammonia water and hydrochloric acid into the fermenter. The ammonia water used serves as not only a pH adjuster but also a nitrogen source for the growth of E. coli.

In the fed-batch cultivation as described above, as the absorbance (OD₆₀₀) of the culture medium reached 40, 6-8% (w/v) of lactose for the total amount of the culture medium was dissolved until it becomes 1 liter and was added to be expressed for 10 hours, thus producing the T-20 peptide. The expression step was performed at 37° C. and pH 6.8-7.2 under atmospheric pressure.

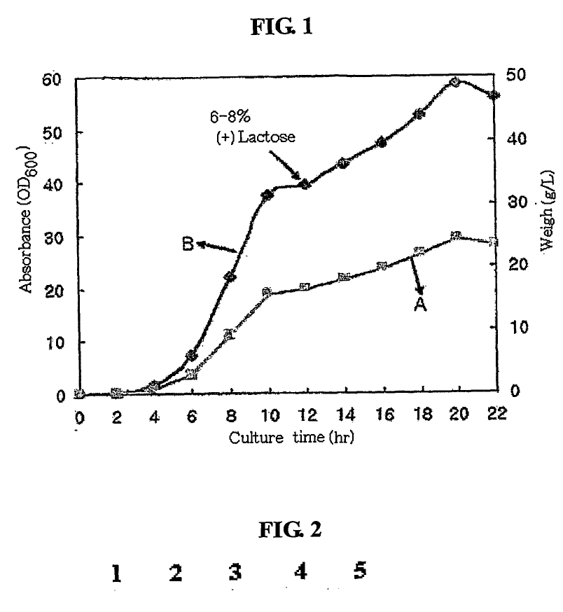

The cultured E. coli broth was centrifuged to isolate E. coli from the culture broth, and the isolated E. coli cells were washed by suspending them in distilled water and then centrifuging the suspension. Then, the washed E. coli cells were dried in a dryer at 60° C. for 34 hours, and their weight was measured. The measurement results are shown in FIG. 1. As shown in FIG. 1, the growth rate of the recombinant E. coli with the passage of culture time was measured to be a crude protein content of more than 55% and a nucleic acid content of less than 8% at a growth rate of less than 0.1 hr⁻¹. The dry cell weight after completion of the fermentation was 23.3 g/L.

Moreover, in order to examine whether the T-20 peptide have been expressed, samples sampled with the passage of time after the expression were washed in the same manner as described above, suspended in a pH 8.0 buffer solution and electrophoresed in 16.2% tricine gel. The electrophoresis results are shown in FIG. 2. As shown in FIG. 2, it could be found that a 6.5 kDa fusion protein was expressed in the form of a T-20 peptide-fusion partner. The amount of the T-20 peptide was about 20% of the total protein, and yield was about 1 g/liter.

### Example 2

### Isolation and Purification of T-20 Peptide

In order to isolate the T-20 peptide from the E. coli cells cultured in Example 1,100 g (wet weight) of the microbial cells were suspended in distilled water so as to obtain 400 ml of a suspension, which was then stirred. The suspension was treated with 600 ml of acetone and well stirred. The stirred solution was centrifuged, and the supernatant was collected and treated with five times as much amount of acetone which had been cooled at −20° C. as the supernatant. As a precipitate was formed, the solution was centrifuged again, and the precipitate was collected and dried.

In order to cut out the fusion partner from the isolated fusion protein of the T-20 peptide-fusion partner, the fusion protein was treated with CNBr. In this step, after 4 g of dried precipitate was dissolved in 100 ml of 70% formic acid the resulting solution was treated with 6N CNBr to be 2N. Then, the solution was allowed to react for 24 hours. After completion of the reaction, the solution was treated with acetone, and the formed precipitate was collected and dried.

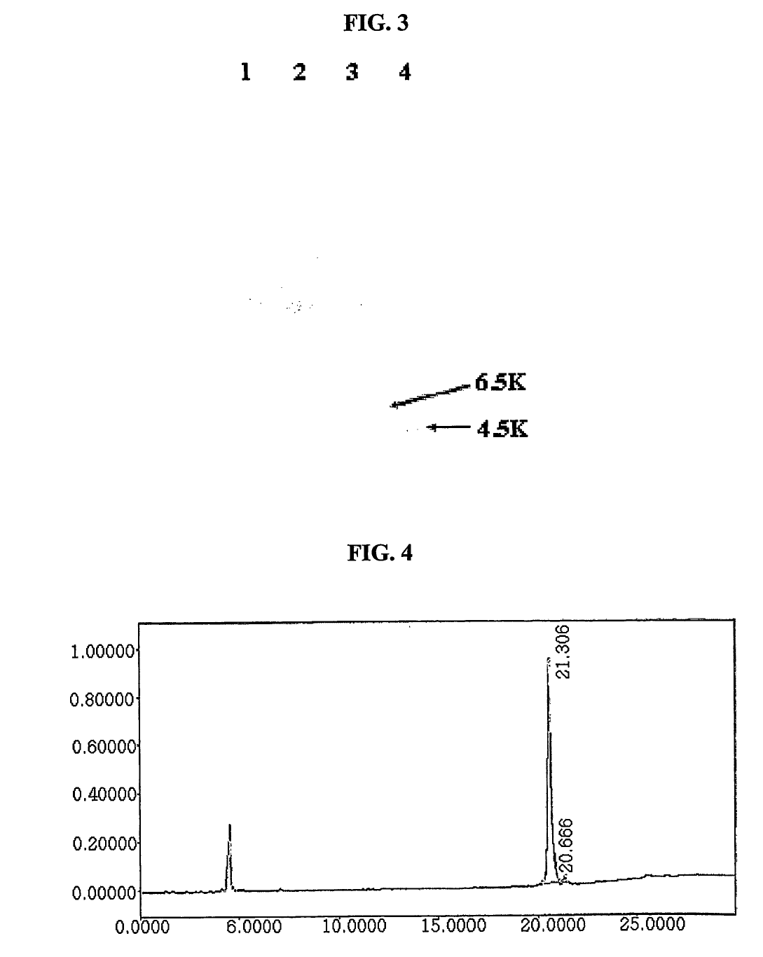

The dried precipitate was dissolved in 1 ml of 200 mM sodium carbonate solution to be 90 mg and adjusted to pH 8.0 with 2N hydrochloric acid. The resulting solution was adsorbed onto a Sepharose column, which was then eluted at least two times with a salt concentration gradient of 0˜0.5M of a sodium chloride-containing buffer solution. Next, the T-20 peptide in the solution was collected using a 0.4M sodium chloride-containing buffer solution and treated with acetone, and the formed precipitate was dried. The dried precipitate was subjected to high-pressure column packed with C18 so as to completely remove impurities, and the resulting material was treated with acetone in the same manner. The formed precipitate was collected and dried. The dried precipitate obtained by the purification as described above was electrophoresed. The electrophoresis results are shown in FIG. 3. As shown in FIG. 3, it could be found that about 4.5-kDa T-20 peptide was obtained.

Furthermore, the resulting dried material was subjected to HPLC analysis. As shown in FIG. 4 and Table 1, it could be found that the T-20 peptide having no ion peak was obtained with high purity. Also, the molecular weight of the resulting dried material was analyzed by Q-Top MS/MS, and the results showed that the molecular weight was 4.45 kDa which is the same as that of the T-20 peptide.

TABLE 1

| # | RT (min) | Area (mV*sec) | Type | Width (sec) | Area (%) |
|---|---|---|---|---|---|
| 1 | 20.565 | 12350.745 | BB | 56.300 | 99.144 |
| 2 | 21.305 | 106.586 | BB | 14.300 | 0.856 |

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

### INDUSTRIAL APPLICABILITY

As described above, the present invention provides the method for preparing the T-20 peptide by the high-cell-density cultivation of recombinant E. coli containing the T-20 peptide-coding gene, as well as the method by which the T-20 peptide is isolated and purified with high purity from E. coli which had been cultured with high cell density and in which the T-20 peptide have been expressed. Thus, the present invention provides the method for economically preparing the T-20 peptide with high industrial value.

1. A method for preparing a T-20 peptide by the high-cell-density culture of recombinant E. coli containing a T-20 peptide-coding gene, the method comprising the steps of:
(a) batch-culturing recombinant E. coli containing the T-20 peptide coding gene in an initial culture medium (pH 6.8-7.2) containing mineral solution;
(b) culturing the recombinant E. coli while adding a nutrient medium automatically so as to adjust the pH of the culture broth to 6.8-7.2; and
(c) adding an expression inducer when the absorbance (OD₆₀₀) of the recombinant E. coli culture broth reaches 30-100, and culturing the recombinant E. coli additionally so as to induce the expression of the T-20 peptide.

2. The method for preparing a T-20 peptide according to claim 1, wherein the recombinant E. coli containing the T-20 peptide coding gene is E. coli BmG3 (KCCM 10506).

3. The method for preparing a T-20 peptide according to claim 1, wherein the expression inducer is lactose.

4. The method for preparing a T-20 peptide according to claim 3, wherein the concentration of lactose is 6˜8% (w/v).

5. A method for isolating and purifying a T-20 peptide, the method comprising the steps of:
(a) collecting the E. coli where the T-20 peptide, produced by the method of any one claim among claims 1 to 4, have been expressed in a form fused with a fusion partner, and then suspending the collected E. coli in distilled water;
(b) treating the recombinant E. coli suspension with an organic solvent, to obtain a precipitate containing a fusion protein in the form of the T-20 peptide-fusion partner;
(c) cutting out the fusion partner from the fusion protein, and then treating the remaining protein with an organic solvent so as to obtain a precipitate containing the T-20 peptide; and
(d) subjecting the precipitate to chromatography so as to remove impurities, treating the remaining protein with an organic solvent, and collecting the treated protein.

6. The method for isolating and purifying a T-20 peptide according to claim 5, wherein the concentration of organic solvent added in the suspension in the step (b) is 10˜60%.

7. The method for isolating and purifying a T-20 peptide according to claim 5, wherein the organic solvent is acetone.

8. The method for isolating and purifying a T-20 peptide according to claim 5, wherein the step (c) comprises the sub-steps of:
(i) adsorbing the precipitate solution onto a Sepharose column, eluting the Sepharose column at least two times with a concentration gradient of sodium chloride, collecting the resulting precipitate, and precipitating the precipitate by treating with acetone; and
(ii) subjecting the precipitate obtained in the step (i) to C18 column chromatography so as to remove impurities, and precipitating the remaining substance by treatment with acetone.

9. The method for isolating and purifying a T-20 peptide according to claim 8, wherein the buffer solution containing sodium chloride is used for collecting the resulting precipitate in the step (i).

10. The method for isolating and purifying a T-20 peptide according to claim 5, wherein the cutting out the fusion partner from the fusion protein in the step (c) is treated with CNBr.

11. The method for isolating and purifying a T-20 peptide according to claim 6, wherein the organic solvent is acetone.
